(19) United States
(12) Patent Application Publication
(10) Pub. No.: US 2020/0135712 A1
(43) Pub. Date: Apr. 30, 2020

(54) IMPLANTABLE DEVICE AND MANUFACTURING METHOD OF THE SAME

(71) Applicant: SEOUL NATIONAL UNIVERSITY R&DB FOUNDATION, Seoul (KR)

(72) Inventors: Yoon Kyu SONG, Seoul (KR); Jung Woo JANG, Seoul (KR); Chae Eun LEE, Seoul (KR)

(21) Appl. No.: 16/663,436

(22) Filed: Oct. 25, 2019

(30) Foreign Application Priority Data
Oct. 25, 2018 (KR) 10-2018-0128119

(51) Int. Cl. H01L 27/02; H01L 27/08; H01L 29/861; H01L 29/66; A61B 5/00

(52) U.S. Cl. CPC H01L 27/0248; H01L 27/0814; A61B 5/6847; H01L 29/66128; H01L 29/66136; H01L 29/8611

(57) ABSTRACT

Disclosed is an implantable device including: a first insulation layer; a second insulation layer arranged on the first insulation layer; a first semiconductor layer arranged between the first and second insulation layers; a second semiconductor layer doped into the first semiconductor layer, with the second semiconductor layer forming a closed loop as seen in a top view; a metal layer disposed on the second insulation layer, with the metal layer forming an electrode; a third insulation layer covering the metal layer; and an insulation region including the first and second semiconductor layers.

### CROSS REFERENCE TO RELATED APPLICATION

This application is claims the benefit and priority of Korean Patent Application No. 10-2018-0128119, filed on Oct. 25, 2018. The entire disclosures of the applications identified in this paragraph are incorporated herein by reference.

### FIELD

The present invention relates generally to an implantable medical device and a manufacturing method of the same, and more particularly, to an implantable medical device and a method for manufacturing the same by incorporating a CMOS process.

### BACKGROUND

This section provides background information related to the present disclosure which is not necessarily prior art.

FIGS. 1A and 1B illustrate an example of an optical probe LED chip module for bio stimulation and a manufacturing method of the same described in Korean Patent Application Publication No. 10-2013-0078956.

The optical probe LED chip module 1 comprises an LED chip 10, a substrate 30 adapted to support the LED chip 10, an optical waveguide 50 adapted to collect light emitted by the LED chip 10, and an insulator 70 adapted to connect the optical waveguide 50 and the substrate 30 together and to insulate them from the outside. The optical waveguide 50 includes a cylindrical, elongated body 51 having one side facing the LED chip 10, a transformation zone 53 having a gradually diminishing diameter away from the other side of the body 51, and a probe 55 extended from the end of the transformation zone 53, the probe having a diameter similar to the diameter of an optical fiber.

As shown, the optical probe LED chip module 1 for bio stimulation is placed in the human brain to stimulate the brain.

The probe 55 of the LED chip module 1 is brought in direct contact with the brain and transmits light to the brain.

The insulator 70 is adapted to connect the substrate 30 and the optical waveguide 50 together, and to optically/mechanically insulate the LED chip module 1 from outside. That is to say, the insulator 70 is responsible for mechanical connection and electrical insulation of the LED chip module 1. It also stops moisture intrusion in the LED chip module 1 and prevents a light leak from the LED chip module 1.

Moreover, the insulator 70 can seal the entire LED chip module 1, except for an area where the LED chip module 1 is connected outside the brain. For instance, the insulator 70 may enclose the LED chip module 1, while exposing a portion of the probe 55 and a portion of an electrode connector 90.

To this end, the insulator 70 is generally made from a material that is safe or harmless to the body, highly light-absorbent, water-proof, and electrically insulating. A good candidate for the material is black epoxy known as a light-absorbent insulating material.

FIG. 2A to FIG. 3F describe an exemplary CMOS process in the prior art.

Sectional views in FIG. 2A to FIG. 2F show changes in a MOSFET along with the basic CMOS process. While the basic CMOS process that is actually used in industries is described herein, it should be noted that a much greater number of process steps are involved. By way of example, in fabrication of NMOSFET and PMOSFET, n-well process for forming an n-well on the p-type silicon substrate may be conducted or p-well process for forming a p-well on the n-type substrate may be conducted, as needed. Now, there is twin-well process for forming both the n-well and p-well on a lightly doped substrate. The use of the twin-well process allows for optimizing PMOSFET and NMOSFET performances and resolving a reliability issue, e.g. latch-up, with the substrate region having low impurity concentration.

First of all, as shown in FIG. 2A, the silicon substrate is oxidized to grow across the entire wafer surface an oxide layer that acts as a barrier during diffusion. Once the oxide layer is grown, a photosensitive polymer called a photoresist is applied across the entire wafer surface and then a portion of the photoresist in an area where an n-well is to be formed is removed with an n-well mask employing a photolithography or photographic process. Referring to FIG. 2B, with the residual photoresist as a barrier material, the oxide layer in the area where an n-well is to be formed is removed by etching. Again, with the residual oxide layer as a barrier material, an n-type impurity such as phosphorous (P) is diffused to form an n-well, as shown in FIG. 2C.

Referring now to FIG. 2D, part of the residual oxide layer is removed. Next, a pad oxide layer for protecting a silicon interface is grown across the entire wafer surface by oxidation, and a silicon nitride (Si₃N₄) is formed on the pad oxide layer by low-pressure chemical vapor deposition, as shown in FIGS. 2E and 2F. Although not shown, an active area where a translator is fabricated is later defined by preserving a laminated structure of the pad oxide layer and the nitride layer on the active area only, which is accomplished by employing an active area mask and the photolithography and etching processes. With the residual laminated structure as a barrier, boron (B) ions are implanted into the p-type substrate area and phosphorous (P) ions are implanted into the n-well region, respectively, such that the concentration of impurities in areas outside the transistor will increase. As a result, any undesired channel would not be formed in the areas outside the transistor in operation of the circuit. Accordingly, this step is called a channel-stop implantation process. In order to perform the channel-stop implantation process separately on the p-type substrate region and the n-well region, boron ions are implanted into the p-type substrate region using the aforementioned n-well mask and photolithography, so that the photoresist on the n-well region is preserved and protected. Similarly, phosphorous ions are implanted into the n-well region, and the p-type substrate region is protected as the photoresist thereon is preserved. High-temperature oxidation is immediately followed for an extended period of time to yield a thick oxide layer across the entire region except for the active area. This oxide layer serves as an isolator between each translator. The pad oxide layer and the nitride layers are all removed.

Referring now to FIG. 2E, a thin oxide layer is formed by oxidation to fabricate a MOSFET in the active area. This oxide layer is used as a gate oxide of the MOSFET. Therefore, it is important to obtain a high-quality oxide layer in this step in order to obtain a desirable MOSFET. Following the formation of the gate oxide, an ion implantation process is carried out in such a way that a threshold voltage of the MOSFET is properly adjusted. This is called a threshold voltage adjustment implantation process. For independent adjustment of threshold voltage of the NMOSFET and PMOSFET, the photoresist is used to selectively implant ions only into desired areas. A polysilicon layer used as a gate material is then formed across the entire wafer surface by chemical vapor deposition. Next, the polysilicon layer is removed through a gate mask and the photolithography and etching processes, except for those portions of the polysilicon layer on a gate of the MOSFET and on a part used as a connecting conductor between elements (see FIG. 3A).

Once the gate is obtained as described above, phosphor ions are implanted into the NMOSFET and boron ions are implanted into the PMOSFET, respectively, at a high concentration, to form sources and drains as shown in FIG. 3B. Again, the photoresist is used to selectively implant these ions only into desired areas. In the formation of sources/drains, the polysilicon gate acts as a barrier material for preventing ion implantation into an area under the gate. This process is called a self-align process as the gate and the sources/drains are aligned spontaneously, using the polysilicon as a gate material. Optionally, the ions implanted through a high-temperature diffusion process may be distributed deeper below the surface of the substrate.

Referring now to FIG. 3C, a thick nitride layer is deposited across the entire surface in order to isolate the polysilicon layer used as the connecting connector between elements from a metal layer which will be deposited on top of the polysilicon layer. The nitride layer is selectively etched with a mask on areas where the metal layer is connected to the element, as shown in FIG. 3D. Next, an evaporation or sputtering process is conducted to deposit the metal layer as shown in FIG. 3E. The metal layer is etched with a mask having desired connections to obtain a structure in FIG. 3F.

Lastly, although not shown, a passivation layer for protecting a chip may be deposited, and a bonding pad which is to be connected by a lead frame and thin wire of a package used for packing the chip as an end product is etched with a mask. The chip thus completely manufactured is tested and packaged before it is placed on the market.

FIGS. 4A and 4B illustrate an implantable biometric chip for checking vitals through a micro semiconductor chip inserted in the body, as described in Korean Patent Application Publication No. 10-2018-0069319.

FIG. 4A shows an exemplary embodiment of an implantable biometric chip. It is arranged in the left or right side the carotid artery, without any functional difference depending on its position. There are two sensors outside the chip, including a GPS sensor for finding out the location of a patient, and a temperature sensor for detecting an internal temperature.

When the chip located in the carotid artery obtains a vital signal inside the body, an alarm sensor at the center of the chip responds to it and sends a signal to monitors present in a fire station and ER waiting room. Emergency bells installed at the monitors are sounded and red light thereof is turned on. The current location and condition of the patient can be identified and analyzed through the monitors.

FIG. 4B is an exploded view of the implantable biometric chip. It shows major components of the chip. The chip is comprised of biologically safe packaging.

There are two sensors with primary functions in the chip. One of them is an ultrasonic sensor for getting an ECG. The other is a pressure sensor for measuring blood pressure on a regular basis to check an abnormal pulse rate.

The biometric chip is surrounded with a packaging 7, and the sensors, i.e., the temperature sensor 3, the pressure sensor 4, the ultrasonic sensor 5 and the GPS sensor 6 are mounted in the packaging 7. The current condition of the patient obtained by the GPS sensor 6 and the ultrasonic sensor 5 is notified to the emergency bell 1 and a monitor 2 for analysis. The monitor 2 for analysis is designed to help healthcare providers to find out the current location and health condition of a wearer or patient of the biometric chip and to provide the patient with emergency assistance. The emergency bell 1 gives an immediate notice to the biometric chip wearer if any emergency occurs. The packaging 7 surrounding the biometric chip can be made of a material that is biologically safe and harmless to the body and has a long lifespan. All information on the current condition obtained by the biometric chip can be stored frequently in a record storage unit 9. The ultrasonic sensor 5 checks ECGs regularly, and if the ECG reading is abnormal the ultrasonic sensor 5 notifies it to an alarm generator 10. In an emergency situation, the alarm generator 10 sends the results concerning the patient's location and condition to the emergency bell 1 and the monitor 2 for analysis. Chips in the biometric chip are composed of a wafer 8 featuring excellent reliability and stability.

In general, because the implantable device or chip as illustrated in FIG. 1 and FIG. 4 are electrically operated, its lifespan can be shortened by frequency contacts with body fluid. Therefore, it is preferable that the device or chip is isolated from the body fluid. Although there are different types of resin-based materials for insulation, epoxy is not desirable as it does not completely block moisture.

Those chips manufactured by the CMOS process illustrated in FIGS. 2 and 3 are not free from insulation problems inside the body. Because of this, a separate insulation process should be conducted on an end product. In other words, a separate insulation process needs to be conducted after the general CMOS process is all over.

### SUMMARY

The present disclosure is directed to an insulating implantable device manufactured in typical CMOS processes, in which the implantable device is made of a material of an excellent moisture blocking property, instead of a resin-based material.

According to one aspect of the present disclosure, there is provided an implantable device including: a first insulation layer; a second insulation layer arranged on the first insulation layer; a first semiconductor layer arranged between the first and second insulation layers; a second semiconductor layer doped into the first semiconductor layer, with the second semiconductor layer forming a closed loop as seen in a top view; a metal layer disposed on the second insulation layer, with the metal layer forming an electrode; a third insulation layer covering the metal layer; and an insulation region including the first and second semiconductor layers.

According to another aspect of the present disclosure, there is provided a manufacturing method of an implantable device, the method including: preparing a supporting substrate having a first insulation layer; arranging a first semiconductor layer on the first insulation layer; forming a second semiconductor layer within the first semiconductor layer to form a closed loop as seen in a top view; forming a second insulation layer within the first semiconductor layer; forming, on the second insulation layer, a metal layer having an electrode; forming a third insulation layer on the metal layer; and removing the supporting substrate.

These and other advantages of the present invention will be recognized by those of skill in the art in view of the following detailed description, the accompanying drawings and the appended claims.

### BRIEF DESCRIPTION OF THE DRAWINGS

FIGS. 1A and 1B illustrate an example of an optical probe LED chip module for bio stimulation and a manufacturing method of the same described in Korean Patent Application Publication No. 10-2013-0078956.

FIGS. 2A, 2B, 2C, 2D, 2E, 2F, 3A, 3B, 3C, 3D, 3E and 3F describe an exemplary CMOS process in the prior art.

FIGS. 4A and 4B illustrate an implantable biometric chip for checking vitals through a micro semiconductor chip inserted in the body, as described in Korean Patent Application Publication No. 10-2018-0069319.

FIGS. 5A and 5B illustrate an exemplary embodiment of an implantable device according to the present disclosure.

FIGS. 6A, 6B, 6C, 6D, 6E, 6F and 6G show an exemplary embodiment of a manufacturing method of an implantable device according to the present disclosure.

FIGS. 7A and 7B show another exemplary embodiment of a manufacturing method of an implantable device according to the present disclosure.

FIG. 8 shows another exemplary embodiment of a manufacturing method of an implantable device according to the present disclosure.

FIG. 9 shows another exemplary embodiment of a manufacturing method of an implantable device according to the present disclosure.

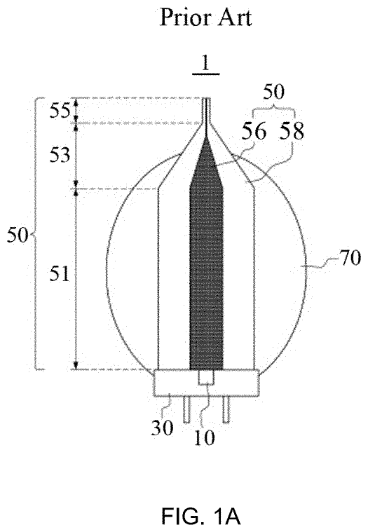

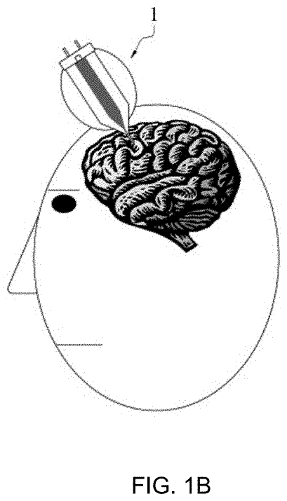

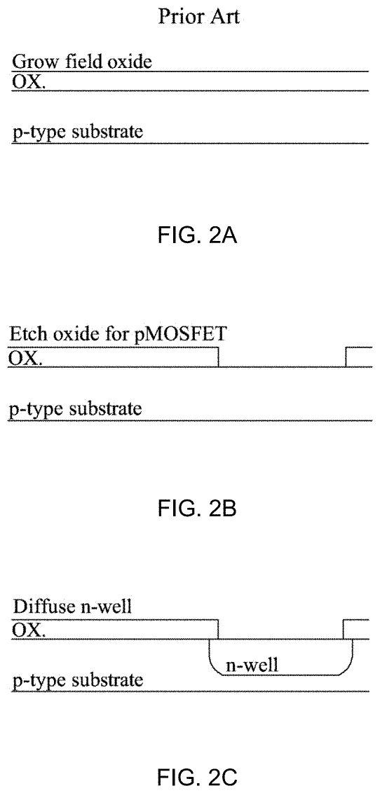

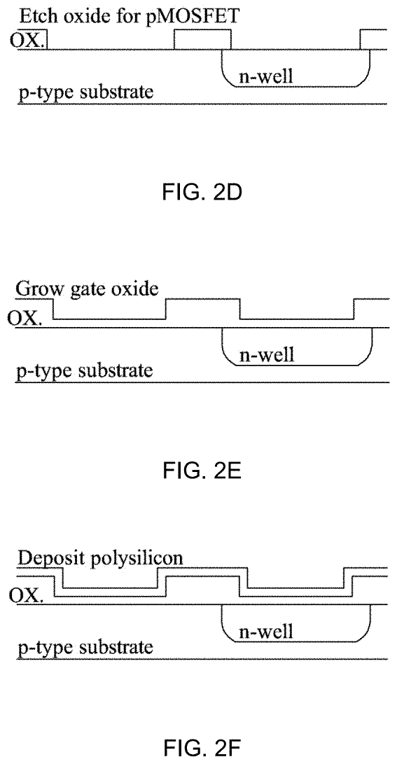

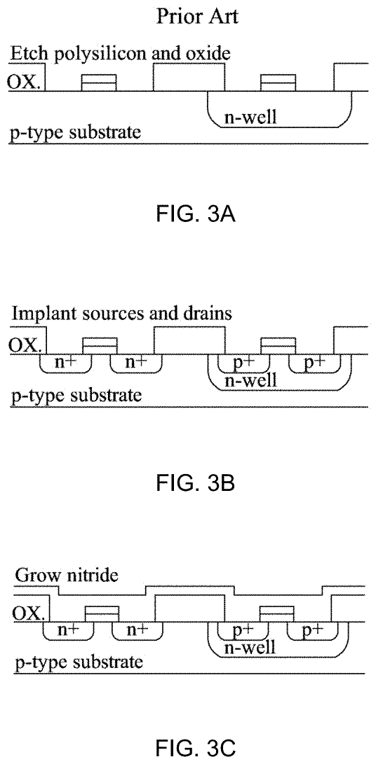

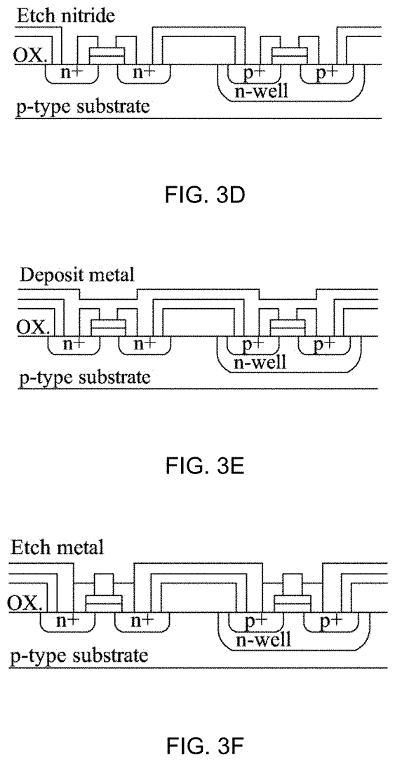

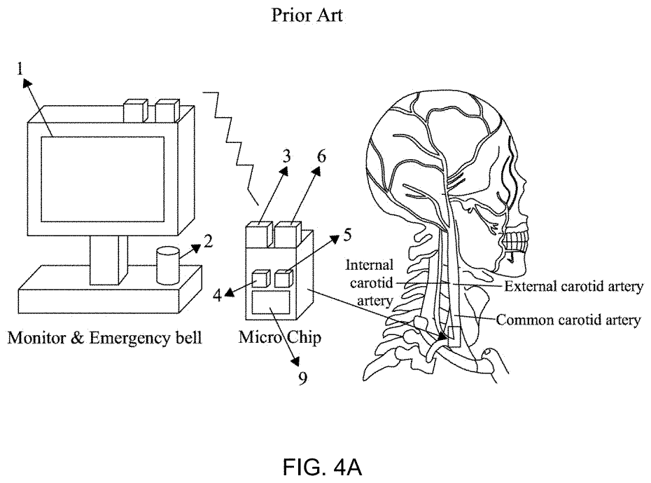

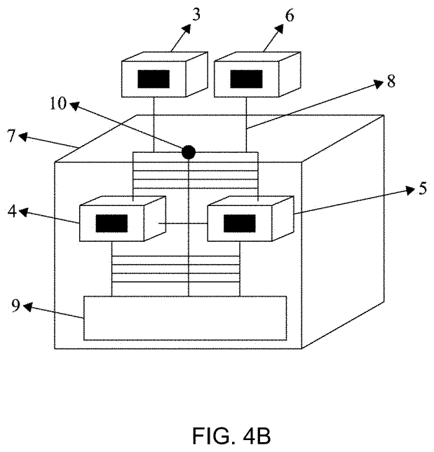

### DETAILED DESCRIPTION

The present disclosure will now be described in detail with reference to the accompanying drawing(s).

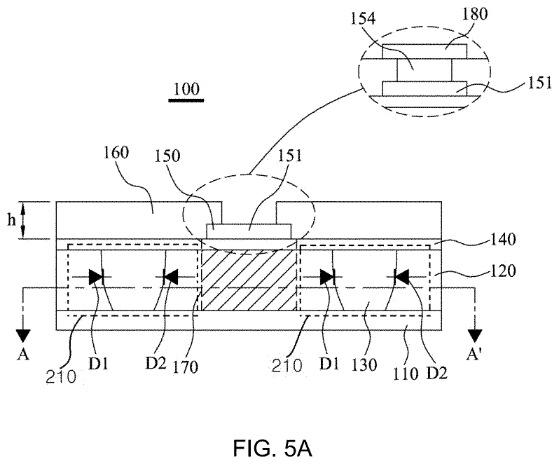

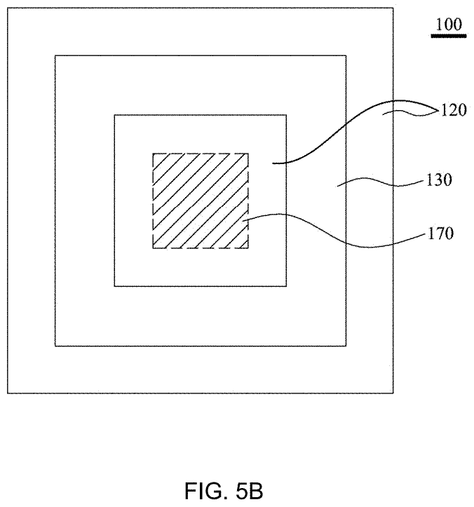

FIGS. 5A and 5B illustrate an exemplary embodiment of an implantable device 100 according to the present disclosure.

FIG. 5A is a sectional view of the implantable device 100, and FIG. 5B is a top view with a second insulation layer 140 and a third insulation layer 160 being removed from FIG. 5A.

The implantable device 100 includes a first insulation layer 100, the second insulation layer 140, a first semiconductor layer 120, a second semiconductor layer 130, a metal layer 150 and the third insulation layer 160.

The first insulation layer 110 is disposed at the very bottom of the device 100. The first insulation layer 110 can be an oxide layer. For instance, the first insulation layer 110 can be SiO₂. The second insulation layer 140 is arranged on the first insulation layer 110, and can be SiO₂, for example.

The first semiconductor layer 120 is arranged between the first insulation layer 110 and the second insulation layer 140. The second semiconductor layer 130 is formed within the first semiconductor layer 120 in such a manner that the second semiconductor layer 130 forms a closed loop in the first semiconductor layer 120 as seen in the top view. The first semiconductor layer 120 and the second semiconductor layer 130 have the same height on the sectional view. The upper face of the first semiconductor layer 120 and the upper face of the second semiconductor layer 130 come in contact with the second insulation layer 140. The lower face of the first semiconductor layer 120 and the lower face of the second semiconductor layer 130 come in contact with the first insulation layer 110. The second insulation layer 140 can abut onto an insulation region 210. The first semiconductor layer 120 and the second semiconductor layer 130 may be present in the insulation region 210.

The metal layer 150 may be isolated from the isolation region 210. Also, the metal layer 150 may be in electrical communication with a device region 170.

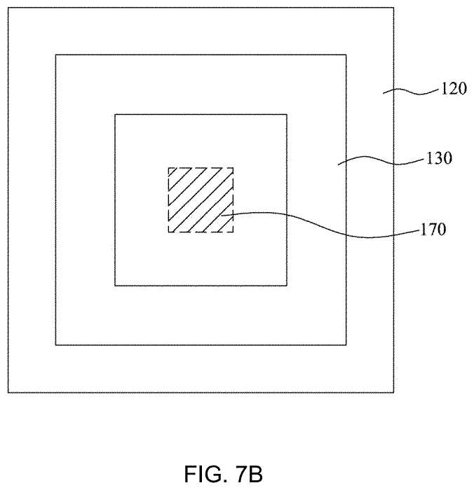

The device 100 may further include an electrode 151 for external stimulation on the second insulation layer 140. As seen in the top view, the electrode 151 is located inside the closed loop. The electrode 151 is an exposed portion of the metal layer 150 by the third insulation layer 160. The metal layer 150 may be arranged in the device region 170 and connect elements therein. The exposed electrode 151 may be made of platinum. In particular, the metal layer 150 may be a stack of layers, with only the uppermost, exposed electrode 151 being formed of platinum. More details on the device region 170 will be provided later with reference to FIG. 7. The device region 170 can be present inside the closed loop, as seen in the top view.

The third insulation layer 160 covers the metal layer 150. The third insulation layer 160 may include at least one layer. Preferably, the third insulation layer 160 has a height of at least 1 μm and not greater than 20 μm. Further, the third insulation layer 160 is connected to the electrode 151 and may have a pad 180 for external stimulation. The pad 180 is electrically connected to the metal layer 150, passing through the third insulation layer 160. To this end, an electrical connection 154 may be arranged between the pad 180 and the metal layer 150. For instance, this electrical connection 154 may be formed of copper (Cu). The third insulation layer 160 may be SiO₂. Preferably, the pad 180 is made of a material that does not react with body fluid. One example of such material is platinum.

The metal layer 150 can be obtained by the standard pad process in typical CMOS processes. If needed, however, the metal layer 150 can be made thinner.

As shown in FIG. 5, for example, if the first semiconductor layer 120 is p-type and the second semiconductor layer 130 is n-type, a PN junction is created between the first semiconductor layer 120 and the second semiconductor layer 130. As such, lateral faces of the first semiconductor layer 120 can be insulated. Without limiting to the example in FIG. 5, it is also possible that the first semiconductor layer 120 is n-type and the second semiconductor layer 130 is p-type. Still, the PN junction therebetween allows the corresponding semiconductor layer to withstand several tens of volts or even static electricity. Moreover, when diodes D₁ and D₂ are arranged facing each other as shown in FIG. 5 and voltage runs in reverse direction around the second semiconductor layer 130, current will not flow in any direction. Accordingly, the device region 170 is protected from an externally applied current.

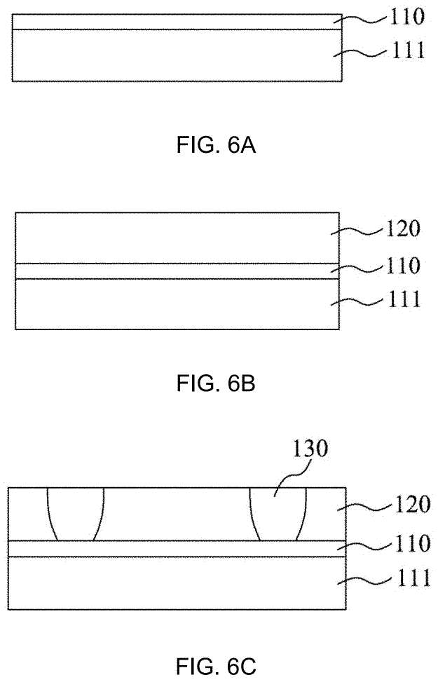

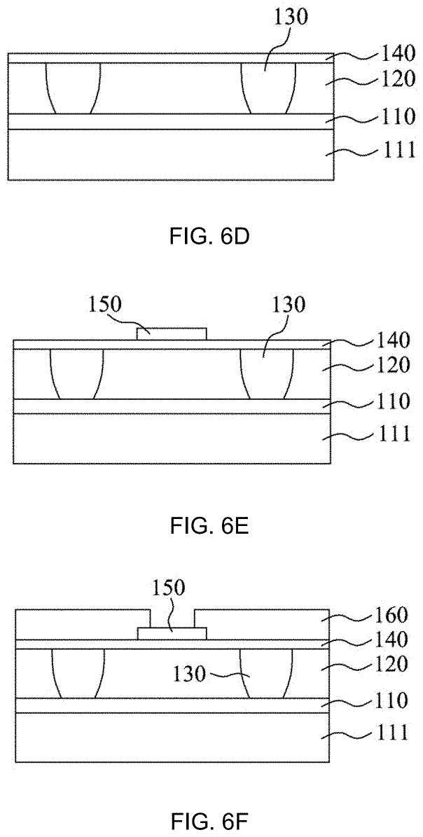

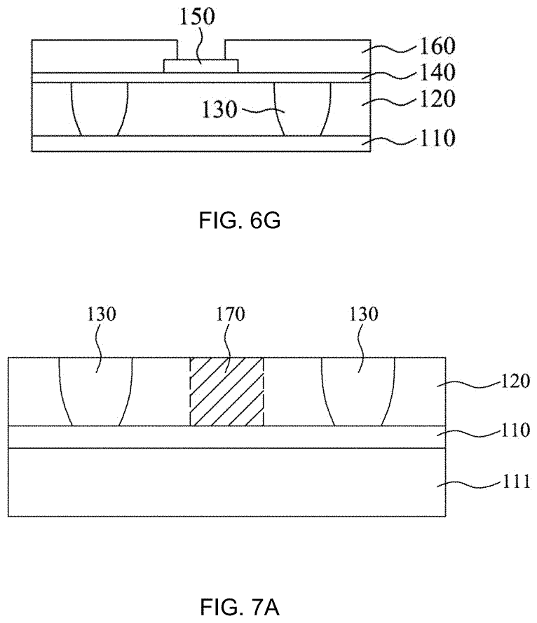

FIG. 6A to FIG. 6G describe an exemplary embodiment of a manufacturing method of an implantable device 100 according to the present disclosure.

In order to manufacture the implantable device 100, a supporting substrate 111 having a first insulation layer 110 is prepared as shown in FIG. 6A. The supporting substrate 111 may be a silicon wafer, and the first insulation layer 110 may be an oxide layer obtained by oxidation of the silicon wafer.

Next, a first semiconductor layer 120 is arranged on the first insulation layer 110, as shown in FIG. 6B. The first semiconductor layer 120 can be bonded to the first insulation layer 110.

Referring now to FIG. 6C, a second semiconductor layer 130 is formed in the first semiconductor layer 120, forming a closed loop as seen in the top view. The second semiconductor layer 130 can be obtained by implanting impurities into the first semiconductor layer 120. Preferably, the second semiconductor layer 130 is formed within the first semiconductor layer 120 and serves to divide the first semiconductor layer 120 into inside and outside parts. Accordingly, in an insulation region 210, the first semiconductor layer 120 and the second semiconductor layer 130 are repeated at least once within the first semiconductor layer 120 and a PN junction is created to prevent electricity from flowing to a device region 170.

As shown in FIG. 6D, a second insulation layer 140 is then formed on the first semiconductor layer 120.

Referring next to FIG. 6E, a metal layer 150 including an electrode 151 is formed on the second insulation layer 140. For instance, the metal layer 150 may be adapted to connect elements in the device region 170.

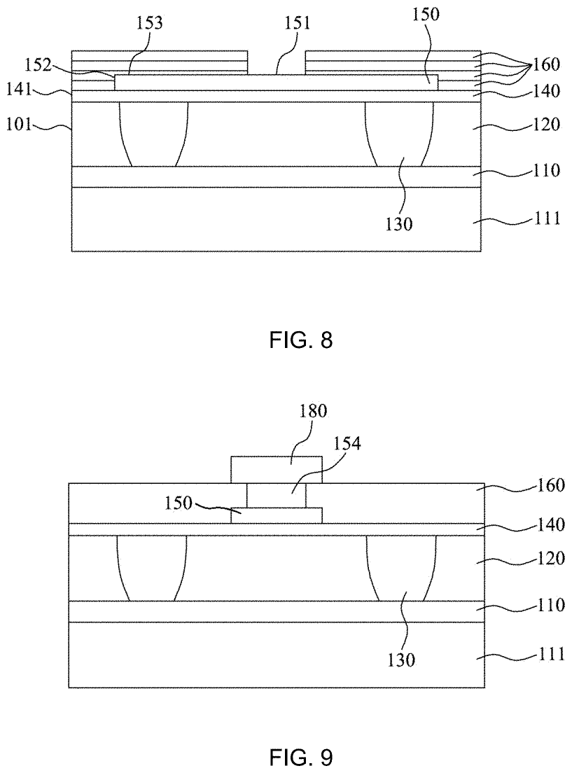

A third insulation layer 160 is formed on the metal layer 150, as shown in FIG. 6F. Subsequent steps after the step shown in FIG. 6F in the process can be repeated multiple times. This will be described in further detail with reference to FIG. 8.

The steps shown in FIGS. 6E and 6F can be repeated several times even in general CMOS process. As such, a plurality of the metal layers 150 can be formed in-between the third insulation layer 160. However, since insulation of lateral faces of the device 100 degrades by an increasing number of the metal layer 150, it is not desirable to have a stack of multiple metal layers 150.

The supporting substrate 111 is removed, as shown in FIG. 6G. The supporting substrate 111 can be etched by an etching solution, for example.

FIGS. 7A and 7B show another exemplary embodiment of a manufacturing method of an implantable device according to the present disclosure.

The second insulation layer 140 is formed on the first semiconductor layer 120 as described above with reference to FIG. 6D. In this alternative embodiment, prior to the step in FIG. 6D, the device region 170 can be formed inside the closed loop as seen in the top view. The device region 170 can be designed for charging, communication, signal measurement or stimulation signaling of the implantable device 100. For example, since the implantable device 100 is insulated from outside, the device region 170 can be adapted to receive power and data wirelessly using a coil-form antenna, to wirelessly send data to an external device, to measure input electrical signals as well as types of light through a loop composed of photodiodes, and to stimulate cells through electric current, voltage or LED light.

FIG. 8 shows another exemplary embodiment of a manufacturing method of an implantable device according to the present disclosure.

In this alternative embodiment, the metal layer 150 is formed further inside than lateral faces 141 of the second insulation layer 140 in the step of FIG. 6E. That is to say, lateral faces 152 of the metal layer 150 are preferably not exposed to lateral faces 101 of the implantable device 100. Moreover, in the step of FIG. 6F, the third insulation layer 160 covers the metal layer 150 in such a way that an upper face 153 of the metal layer 150 is preferably not exposed but covered with the third insulation layer 160, while the electrode 151 of the metal layer 150 can be exposed.

The third insulation layer may be formed of a stack of multiple layers. This is done for better insulation from outside by multiplying the standard height used in typical CMOS processes. For instance, the third insulation layer 160 having a height of about 100 Å is usually formed at a time during the CMOS process. Alternatively, however, a plurality of third insulation layers 160 can be stacked, or the third insulation layer 160 having a micrometer thickness can be formed at a time.

FIG. 9 shows another exemplary embodiment of a manufacturing method of an implantable device according to the present disclosure.

In this alternative embodiment, in the step of FIG. 6F, the metal layer 150 is etched to expose its top face. Next, the pad 180 can be formed on the metal layer 150 and an electrical connection 154 may be arranged between the metal layer 150 and the pad 180 to electrically connect them, as shown in FIG. 9.

While FIG. 9 shows that the supporting substrate 111 is in a bonded state, the subsequent steps in FIG. 9 can still be conducted after the step of removing the supporting substrate 111 in FIG. 6G.

Set out below are a series of clauses that disclose features of further exemplary embodiments of the present disclosure, which may be claimed.

(1) An implantable device comprising: a first insulation layer; a second insulation layer arranged on the first insulation layer; a first semiconductor layer arranged between the first and second insulation layers; a second semiconductor layer doped into the first semiconductor layer, with the second semiconductor layer forming a closed loop as seen in a top view; a metal layer disposed on the second insulation layer, with the metal layer forming an electrode; a third insulation layer covering the metal layer; and an insulation region including the first and second semiconductor layers.

(2) There is also provided, the implantable device of clause (1) wherein: the metal layer is formed further inside the second semiconductor layer as seen in the top view.

(3) There is also provided, the implantable device of clause (1) further comprising: a device region arranged between insulation regions.

(4) There is also provided, the implantable device of clause (1) wherein: the third insulation layer is removed to expose the electrode.

(5) There is also provided, the implantable device of clause (1) wherein: the electrode is formed of platinum.

(6) There is also provided, the implantable device of clause (1) wherein: at least one of the first, second or third insulation layer is SiO₂.

(7) There is also provided, the implantable device of clause (1) wherein: the first and second semiconductor layers have the same height as seen in a sectional view.

(8) There is also provided, the implantable device of clause (1) wherein: the insulation region has both a diode and an inverse diode.

(9) A manufacturing method of an implantable device, the method comprising: preparing a supporting substrate having a first insulation layer; arranging a first semiconductor layer on the first insulation layer; forming a second semiconductor layer within the first semiconductor layer to form a closed loop as seen in a top view; forming a second insulation layer within the first semiconductor layer; forming, on the second insulation layer, a metal layer having an electrode; forming a third insulation layer on the metal layer; and removing the supporting substrate.

(10) There is also provided, the manufacturing method of clause (9) further comprising: prior to forming a second insulation layer on the first semiconductor layer, forming a device region inside the closed loop as seen in the top view.

(11) There is also provided, the manufacturing method of clause (9) wherein: in forming, on the second insulation layer, a metal layer having an electrode, the metal layer is formed inside the second insulation layer as seen in the top view.

(12) There is also provided, the manufacturing method of clause (9) wherein: in forming a third insulation layer on the metal layer, the third insulation layer is formed of a stack of multiple layers.

The implantable device according to the present disclosure has an insulating function achieved in CMOS process.

The manufacturing method of an implantable device according to the present disclosure provides an insulating implantable device by forming the second semiconductor layer within the first semiconductor layer, instead of using a separate insulating material.

What is claimed is:

1. An implantable device comprising:
a first insulation layer;
a second insulation layer arranged on the first insulation layer;
a first semiconductor layer arranged between the first and second insulation layers;
a second semiconductor layer doped into the first semiconductor layer, with the second semiconductor layer forming a closed loop as seen in a top view;
a metal layer disposed on the second insulation layer, with the metal layer forming an electrode;
a third insulation layer covering the metal layer; and
an insulation region including the first and second semiconductor layers.

2. The implantable device according to claim 1, wherein the metal layer is formed further inside the second semiconductor layer as seen in the top view.

3. The implantable device according to claim 1, further comprising:
a device region arranged between insulation regions.

4. The implantable device according to claim 1, wherein the third insulation layer is removed to expose the electrode.

5. The implantable device according to claim 1, wherein the electrode is formed of platinum.

6. The implantable device according to claim 1, wherein at least one of the first, second or third insulation layer is SiO₂.

7. The implantable device according to claim 1, wherein the first and second semiconductor layers have the same height as seen in a sectional view.

8. The implantable device according to claim 1, wherein the insulation region has both a diode and an inverse diode.

9. A manufacturing method of an implantable device, the method comprising:
preparing a supporting substrate having a first insulation layer;
arranging a first semiconductor layer on the first insulation layer;
forming a second semiconductor layer within the first semiconductor layer to form a closed loop as seen in a top view;
forming a second insulation layer within the first semiconductor layer;
forming, on the second insulation layer, a metal layer having an electrode;
forming a third insulation layer on the metal layer; and
removing the supporting substrate.

10. The method according to claim 9, further comprising:
prior to forming a second insulation layer on the first semiconductor layer, forming a device region inside the closed loop as seen in the top view.

11. The method according to claim 9, wherein in forming, on the second insulation layer, a metal layer having an electrode, the metal layer is formed inside the second insulation layer as seen in the top view.

12. The method according to claim 9, wherein in forming a third insulation layer on the metal layer, the third insulation layer is formed of a stack of multiple layers.
